(19) United States
(12) Patent Application Publication
(10) Pub. No.: US 2013/0344171 A1
(43) Pub. Date: Dec. 26, 2013

(54) ACIDIC DIALYSIS CONCENTRATE

(71) Applicant: Manfred Volker, Blankenbach (DE)

(72) Inventor: Manfred Volker, Blankenbach (DE)

(21) Appl. No.: 13/856,793

(22) Filed: Apr. 4, 2013

(30) Foreign Application Priority Data
Apr. 7, 2012 (DE) 102012007165.5

(51) Int. Cl. A61K 33/14; A61K 31/19; A61K 31/7004

(52) U.S. Cl. CPC A61K 33/14; A61K 31/7004; A61K 31/19
USPC 424/678

(57) ABSTRACT

The precursor of an acidic dialysis concentrate is characterized in that two separately provided partial concentrates are formed from the constituents of the acidic dialysis concentrate, wherein only the one partial concentrate contains glucose, preferably glucose monohydrate, while the other partial concentrate comprises other constituents needed for the acidic dialysis concentrate.

### BACKGROUND

Various complications occur in the case of a weak or failing kidney function in the human body. This leads to the concentration increase of several organic substances in the blood. Some of these substances are protein metabolites, i.e. substances which derive their origin from the processing of proteins, such as urea and creatinine. Many of these substances show a toxic effect and are known under the general term toxins. Toxins could be classified according to their molecular size: small molecules below 300 daltons, such as urea (molecular weight MW 60), creatinine (MW 113), medium-sized molecules up to 12,000 daltons, such as parathormone (MW 9424), beta-2-microglobulins (MW 11818), and large molecules, such as myoglobins. These substances serve as markers for acute kidney problems. An induced uremic poisoning will lead to death in the end if these waste products are not removed by way of artificial methods. The most common therapeutic method is hemodialysis, and then peritoneal dialysis.

Dialysis fluid (DF) or dialysate is the liquid that is used during hemodialysis at one side of a membrane within a dialyzer for cleaning the blood of people suffering from kidney failure. The toxins are diffused through small membrane pores from the blood into the DF and the substances from the DF into the blood.

As a rule, the DF solution for hemodialysis is prepared at the place of treatment in a hemodialysis machine from special liquid acid concentrates and a powdery bicarbonate concentrate by proportioning and mixing with treated water of high purity, i.e. an acid concentrate and a base concentrate. Dialysis fluid contains the physiologically most important inorganic cations and anions: sodium, potassium, calcium, magnesium, chlorides, pH adjustment components or buffer (often bicarbonate, acetate, ascorbate, citrate or hydrochloric acid) and an osmotic agent (mostly glucose or icodextrin). One reason for the division of the dialysis concentrate into acidic concentrate and bicarbonate concentrate lies in the low solubility of magnesium and calcium carbonate in the liquid to prevent the precipitation of calcium/magnesium as carbonate salts.

The acid concentrate normally contains sodium chloride, potassium, chloride, calcium chloride, magnesium chloride, glucose, and an acid. The bicarbonate concentrate consists of 1 mol/L solution of sodium bicarbonate and is often provided from powdery sodium bicarbonate.

Concentrate dosage systems for producing DF comprise dosage systems with fixed volumes or dynamic systems. The former use fixed volumes of concentrate and water to form the finished DF. Dynamic dosage systems use pre-set flowmeters often in combination with proportioning pumps for setting the amount of acid and bicarbonate concentrates. The mixing ratio is monitored in both systems by measurement of the conductivity concentration.

In the prior art, the dialysis centers are supplied with liquid, powdery, pasty concentrates, which entails considerable efforts and chemical risks.

It has therefore been the object of the present invention to provide the precursor of a concentrate (super concentrate=SC) particularly for acid concentrates, which corresponds to the objectives listed hereinafter and substantially avoids the disadvantages of the prior art.

1. Low freight and storage costs
1.1 Local preparation of the DF
1.2 Classification of SC as non-dangerous goods

2. Chemical and physical storage stability and medical usability of the finished dialysis solutions

3. Technically easily feasible production process of concentrated mixture of raw components
3.1 Simple quality control for individual constituents
3.2 High solubility efficiency during mixing

4. Technically easily feasible production process of liquid dialysis concentrate

5. Compliance with normative requirements for the finished concentrate without in-house control.

To guarantee low freight and storage costs for the SC, the density of the SC must be as high as possible and the whole volume must be as small as possible. During the storage period there must not be any chemical reaction in the super concentrate that changes the composition of the concentrate. Agglomeration is inter alia to be prevented, so that a homogeneous and complete dissolution is guaranteed.

The SC shall be treated as a non-dangerous substance for international transportation. According to Section 3.3.1 ADR, Special Provision 597, acetic acid solutions with not more than 10% acid content are non-dangerous goods.

Points 3 and 4 refer to a feasible production with means that are as simple as possible for producing SC from individual raw materials, and also to a machine for producing liquid dialysis concentrate from SC in the dialysis centers.

After the production of dialysis concentrate from SC the dialysis concentrate shall meet all normative medical requirements.

The present invention provides suitable means for realizing the listed objectives; the said terms, such as super concentrate (SC), slurry, base concentrate, shall be understood in connection with the respective text passages as a precursor for a hemodialysis concentrate and shall be used accordingly.

Patent Survey

The subject dialysis concentrate preparation has been discussed in several patents.

Patent DE 10 313 965 B3 describes an apparatus for preparing acidic dialysis concentrate. All of the needed substances, except for water, are mixed in a vessel and later diluted with water in situ to obtain dialysis concentrate. There are three drawbacks: relatively complex and expensive mixing-device setup (e.g. integration of the density measurement of the liquid concentrate), glucose powder is stored together with 100% acetic acid in a vessel (problems with chemical stability, point 2). It is also highly debatable whether the vessel with liquid acid can be regarded as a non-dangerous article during transportation (point 1.2).

European patent 0 605 395 B1 refers to a method and an apparatus for preparing a dialysis fluid or a concentrate for producing dialysis fluid or a dialysis concentrate either from powder in the form of a concentrated liquid or powder in dried form or from a slurry with addition of water. The drawback is here the physical and chemical stability of the raw components. It is not taken into account that in the case of a dialysis concentrate preparation from a slurry, some individual constituents (e.g. glucose) require a different pH than the rest of the concentrate, so that a long-term stability is guaranteed.

Moreover, the slurry is not exactly defined (“in the order of 40% by wt.”).

In the apparatus, acid is only added during preparation in liquid or dry powder form in the end product and not together with other substances in the input mixture. For instance in the case of acetic acid, which is volatile, this may lead to an inaccurate concentration. Moreover, the processing of concentrated acetic acid poses problems.

Patent EP 0 456 928 B1 discloses a pasty composition for the dialysis concentrate production which consists of 30-70% of solid electrolyte components (optionally with glucose) and 70-30% of water, and a method for preparing the composition. Sodium acetate, and not acetic acid, is here named as the acetate source. A disadvantage is the poor chemical stability of glucose. Although according to this method all constituents of the acetate-containing dialysis concentrate are prepared in the form of a slurry, glucose is added together with other components. All of this together with a non-optimal pH leads to glucose degradation within the storage period. A classification of this composition as non-dangerous goods is here not indicated.

Patent DE 69 727 811 T2 describes two separate solutions for use in peritoneal dialysis: a glucose-containing solution with a glucose proportion of 20% to 40% at a pH of about 3.2 and a solution with remaining components that are needed for the DF. The solution with the glucose is sterilized and then mixed with the second solution. The method shows a reduced formation of “advanced glycosylation end products (AGEs)”. The patent describes the use of solutions and not of slurries. It is not explained in the patent how the solution with the inorganic salts is prepared—from dry or mixed raw materials with water.

An earlier patent DE 69 230 473 T1 described a similar division for preparing medical DF. A first package contains glucose or a glucose-like compound in a rather small concentration together with further substances; a second package shows a higher glucose content than the first one. The content of the glucose or glucose-like compounds in the second package has a concentration of 40% by wt.

Patent U.S. Pat. No. 6,689,393 B1 uses three solutions for preparing a DF with different glucose concentration. DF solutions with different glucose concentration are often used in peritoneal dialysis and also in so-called profiling (concentration adjustment over time). The first solution contains calcium and electrolyte salts, and optionally glucose in a concentration of less than 0.1M (ca. 18 g/L). The second solution contains glucose in a concentration differing from that of the first solution as well as remaining components in the same concentrations as the first solution. The pH of both solutions is adjusted with an acid to 3.7. A third solution is an alkaline buffer (with bicarbonate, pyruvate, lactate or alpha-ketoglutarate ions). The mixing of all of the three solutions allows the preparation of dialysis fluid with different glucose concentrations. The patent refers to the preparation of dialysis fluid only from true solutions. This will lead to higher storage and transportation costs. Another obvious disadvantage is the relatively complex technical development, setup and maintenance of a mixing unit with three different solutions.

The older patent DE 199 55 578 C1 describes a multi-chamber container for the dialysis concentrate with three compartments for use in kidney dialysis: one for glucose concentrate (more than 10% glucose content, preferably 20-30%, and pH=3), and a second one for sodium, calcium, potassium chloride and a hydrochloric acid instead of acetic acid. Only hydrochloric acid is mentioned as an acidifier. Hydrochloric acid is a strong acid and can have a great influence on the pH buffer. Hydrochloric acid also has the drawback that it is not heat-sterilizable because hydrogen chloride as a gas easily volatizes from the solution. This also causes problems during dosage of the acid in the dialysis concentrate. Hydrochloric acid has a very strong corrosive effect and will pose further problems with respect to production and storage (violation of objective no. 4). It does not become clear from the patent from which physical components (powders, solutions, mixtures) the concentrate is made. When pure raw materials as powders or highly concentrated solutions are used for the preparation of concentrates, increased transportation costs will have to be expected (point 1). Moreover, the intended purpose of a further, substantially empty compartment is not discernible; this leads to increased storage costs.

In patent DE 20 2005 006 624 U1, the suggested raw materials for a dialysis concentrate are distributed in a package unit. A part of the raw materials (K+, Ca2+, Mg2+containing salts, concentrated acetic acid) is prepared in liquid form; sodium chloride and glucose are added in dry form. The main advantage is that it can be easily prepared. Disadvantage: the total volume is not optimized due to the use of dry sodium chloride and glucose because the bulk volume of dry substances is greater than that of moist ones, resulting in increased storage costs. This package unit has a total of four separate containers; this leads to additional technical efforts during mixing (point 4).

A number of patents (e.g. U.S. Pat. No. 6,039,702) describe special containers for sterile medical solutions. The container is an improved variant of a bag (patent WO 93/09820) with 2 or 3 compartments for glucose/glucose-like compound (glucose content at least 10%, pH around 3.5) and the remaining substances. A high pH, high temperature, electrolyte, oxygen and storage time are named as influencing factors for the formation of toxin in the glucose. Both patents deal with dialysis concentrates and not with a composition for producing SC; i.e., in the configuration, the dialysis concentrate is stored separately (glucose and the rest) and not as a slurry.

Patents DE 19 931 077 B4 and DE 10 100 462 B4 disclose methods for producing acidic dialysis concentrate and containers for carrying out the method. The slurry is present in a container and consists of 72-90% raw-material proportion and 28-10% water. The volume is thereby minimized as compared with conventional acidic dialysis concentrates. As an additional advantage, the necessary homogeneity of the concentrate is ensured. One of the drawbacks is that glucose is present together with all components in a very acidic medium at a pH of about 1.4., which is not optimal for avoiding glucose degradation products (shorter storage time). Another drawback in this concentrate formulation is that it is not possible to take reliable samples of glucose for checking quality and composition.

Patents Regarding Dual-Chamber Bags

Patents EP 1 354 607 B1 and DE 10 217 356 describe a formulation for peritoneal dialysis and a dual-chamber bag for dialysis. The first individual solution consists of an osmotic agent (glucose polymer and/or glucose polymer derivative) at a pH ranging from 3.5 to 5.0, preferably 4.2, and of calcium, sodium, magnesium, and chloride ions.

The second solution is a buffer. The dual-chamber bag is a plastic bag with two chambers arranged adjacent to each other. A weld separates the chambers and opens itself upon pressure exerted on the chambers. Differences are

Ready-to-use solutions, no concentrate

Glucose polymer, no glucose

Glucose polymer at pH 4.2 in the first chamber; buffer in the second chamber

No acetate; hydrochloric acid is preferred

Neighboring chamber arrangement

The number of connections is unclear.

Patent DE 3 833 036 C2 describes a double-compartment ampoule with removable closures. After removal of the closures two ampoules are inserted into one another, and the two liquids are mixed. The patent refers to small volumes and is here of no relevance.

Patents DE 69 608 493 T2 and DE 696 062 10 T3 describe a flexible dual-chambered container. The system serves to store a solid and a liquid. Both chambers are constructed with multi-layered sheets to protect from water vapor, oxygen and light beam. The chambers are provided with a breakable or tearable weak seal. The difference with respect to the present package system is

The system is only intended for one solid and one liquid

The chambers are connected side by side

The system is difficult to scale, i.e. to supply in differently sized package units.

Utility model DE 880 376 6 discloses a syringe with a chamber partition wall. Openings between two chambers are opened by operating a rod. The resulting mixture can be pressed out of the syringe by pushing the partition wall. Important features:

This is an application outside hemodialysis

It cannot be automated

The syringe is not suited for mixing large substance volumes.

Another double-chamber ampoule is described in patent DE 103 04 500 A1. This construction has the same drawbacks as the previous utility model.

To achieve the aforementioned objectives, the following mixtures are suggested for the SC.

1. Super concentrate (SC) is divided over 2 parts: in one part, glucose, preferably glucose monohydrate, is mixed with water in the mixing ratio of preferably 2.4:1 (70.6% by wt.: 29.4% by wt.) in the form of a slurry. A small amount of acidic agent (acetic acid, citric acid or other physiologically useable acids) is used for setting the pH in the range 2-3.2 (preferably pH=3.0).

2. The second part of the SC preferably consists of all substances needed for the SC, expect for glucose, namely sodium chloride, potassium chloride, calcium chloride, magnesium chloride or respective hydrates and the rest of the acetic acid. This part is diluted with water also up to the slurry state. The added amount of water should be just great enough to keep the acetic acid concentration, calculated on the basis of the total water content (water from salt hydrate rests plus added water), below 10% in this part of the SC.

3. Chemical composition:

The acid part of the SC (acid base) preferably contains sodium chloride, potassium chloride, calcium chloride dihydrate, magnesium chloride hexahydrate, and acetic acid.

The glucose part of the SC (glucose base) contains glucose and preferably additionally KCl and/or MgCl₂ and/or CaCl₂ and a small amount of acetic acid. The salt amounts are selected such that they can be completely dissolved in a saturated glucose solution at a temperature of about 5° C. The so-called glucose base is set with acetic acid to a pH=3.0.

Both parts of the concentrate have an acetic acid concentration of less than 10% in the liquid phase.

The ratio of solids to liquid in the part acid base is preferably 72% by wt., in the glucose base part preferably 64% by wt. and in the total slurry, i.e. solids+water+acid) about 70% by wt.

The weight ratio of part 1 to part 2 is 4.5 to 5.5:1. The volume ratio of part 1:part 2 is 3.2 to 3.6:1.

Both in part 1 and in part 2 of the SC the respective amounts of potassium chloride, calcium chloride, magnesium chloride or the respective hydrates and the acid can already be pre-diluted with water in conformity with the requirements, and can be supplied in pre-dissolved form to the powdery glucose or the powdery sodium chloride during the production process.

The composition according to the invention for 100 l concentrate and also for the treatment of 1 patient is summarized by way of example in the tables for 1:44 and 1:34 dilution for the purpose of an appropriate application.

The invention provides a precursor of an acidic dialysis concentrate, also called super concentrate (SC) or also slurry above, which is provided as two spatially separated partial concentrates and transported to the place of use. The partial concentrates can be stored over a very long period of time without the glucose being chemically changed or broken down. The transportation volume of the partial concentrates is relatively small because both partial concentrates are present in the form of a slurry with only partly dissolved constituents, said slurries having a reduced volume as dry solids. The partial concentrates of the super concentrate are diluted at the place of use to obtain an acidic dialysis concentrate. Due to the slurry, a high solubility efficiency is achieved during the mixing operation in comparison with dry concentrates. The acidic dialysis concentrate is prepared in the dialysis machines to obtain dialysis fluid.

2-parts composition 1:44 for 100 L acidic concentrate, about 30L(45.843 kg) SLURRY with 74.970 L Aqua purificata

| | NaCl, | KCl, | CaCl2 × 2H2O, | MgCl2 × 6H20, | CH3COOH, | | Glucose × H2O, | Water, | KCl, | MgCl2 × 6H2O, | CH3COOH, |
|---|---|---|---|---|---|---|---|---|---|---|---|
| | kg | kg | kg | kg | kg | Water, L | kg | L | kg | kg | kg |
| Type | Acidic precursor | | | | | | Glucose precursor | | | | |
| Concen- | 27.008 | 0.062 | 0.827 | 0.405 | 0.805 | 8.054 | 4.950 | 2.057 | 0.609 | 0.053 | 0.006 |
| trate 1 | | | | | | | | | | | |
| Concen- | 27.088 | 0.062 | 0.992 | 0.405 | 0.805 | 8.016 | 4.950 | 2.057 | 0.609 | 0.053 | 0.006 |
| trate 2 | | | | | | | | | | | |
| Concen- | 27.088 | 0.397 | 0.992 | 0.405 | 0.805 | 7.862 | 4.950 | 2.057 | 0.609 | 0.053 | 0.006 |
| trate 3 | | | | | | | | | | | |
| Concen- | 27.088 | 0.397 | 0.827 | 0.405 | 0.805 | 7.901 | 4.950 | 2.057 | 0.609 | 0.053 | 0.006 |
| trate 4 | | | | | | | | | | | |
| Concen- | 27.088 | 0.733 | 0.992 | 0.405 | 0.805 | 7.720 | 4.950 | 2.057 | 0.609 | 0.053 | 0.006 |
| trate 5 | | | | | | | | | | | |
| Concen- | 27.088 | 0.733 | 0.827 | 0.405 | 0.805 | 7.746 | 4.950 | 2.057 | 0.609 | 0.053 | 0.006 |
| trate 6 | | | | | | | | | | | |
| Concen- | 27.614 | 0.000 | 0.827 | 0.405 | 1.075 | 7.938 | 4.950 | 2.057 | 0.609 | 0.053 | 0.006 |
| trate 7 | | | | | | | | | | | |
| Concen- | 27.614 | 0.062 | 0.827 | 0.405 | 1.075 | 7.810 | 4.950 | 2.057 | 0.609 | 0.053 | 0.006 |
| trate 8 | | | | | | | | | | | |
| Concen- | 27.614 | 0.397 | 0.827 | 0.405 | 1.075 | 7.682 | 4.950 | 2.057 | 0.609 | 0.053 | 0.006 |
| trate 9 | | | | | | | | | | | |
| Concen- | 27.614 | 0.733 | 0.827 | 0.405 | 1.075 | 7.514 | 4.950 | 2.057 | 0.609 | 0.053 | 0.006 |
| trate 10 | | | | | | | | | | | |

2-parts composition 1:44 for 2.667 L acidic concentrate, about 1.23L(0.87 kg)SLURRY with 1.999 L Aqua purificata

1 patient needs about 1.23 kg slurry during a 5-hour dialysis (dialysate flow about 0.4 L/min)

| | NaCl, | KCl, | CaCl2 × 2H2O, | MgCl2 × 6H20, | CH3COOH, | | Glucose × H2O, | | KCl, | MgCl2 × 6H2O, | CH3COOH, |
|---|---|---|---|---|---|---|---|---|---|---|---|
| | kg | kg | kg | kg | kg | Water, L | kg | Water, L | kg | kg | kg |
| Type | | Acidic precursor | | | | | Glucose precursor | | | | |
| Concen- | 0.722 | 0.002 | 0.022 | 0.011 | 0.021 | 0.215 | 0.132 | 0.055 | 0.016 | 0.001 | 0.00017 |
| trate 1 | | | | | | | | | | | |
| Concen- | 0.722 | 0.002 | 0.026 | 0.011 | 0.021 | 0.214 | 0.132 | 0.055 | 0.016 | 0.001 | 0.00017 |
| trate 2 | | | | | | | | | | | |
| Concen- | 0.722 | 0.011 | 0.026 | 0.011 | 0.021 | 0.210 | 0.132 | 0.055 | 0.016 | 0.001 | 0.00017 |
| trate 3 | | | | | | | | | | | |
| Concen- | 0.722 | 0.011 | 0.022 | 0.011 | 0.021 | 0.211 | 0.132 | 0.055 | 0.016 | 0.001 | 0.00017 |
| trate 4 | | | | | | | | | | | |
| Concen- | 0.722 | 0.020 | 0.026 | 0.011 | 0.021 | 0.206 | 0.132 | 0.055 | 0.016 | 0.001 | 0.00017 |
| trate 5 | | | | | | | | | | | |
| Concen- | 0.722 | 0.020 | 0.022 | 0.011 | 0.021 | 0.207 | 0.132 | 0.055 | 0.016 | 0.001 | 0.00017 |
| trate 6 | | | | | | | | | | | |
| Concen- | 0.736 | 0.000 | 0.022 | 0.011 | 0.029 | 0.212 | 0.132 | 0.055 | 0.009 | 0.001 | 0.00017 |
| trate 7 | | | | | | | | | | | |
| Concen- | 0.736 | 0.002 | 0.022 | 0.011 | 0.029 | 0.208 | 0.132 | 0.055 | 0.016 | 0.001 | 0.00017 |
| trate 8 | | | | | | | | | | | |
| Concen- | 0.736 | 0.011 | 0.022 | 0.011 | 0.029 | 0.205 | 0.132 | 0.055 | 0.016 | 0.001 | 0.00017 |
| trate 9 | | | | | | | | | | | |
| Concen- | 0.736 | 0.020 | 0.022 | 0.011 | 0.029 | 0.200 | 0.132 | 0.055 | 0.016 | 0.001 | 0.00017 |
| trate 10 | | | | | | | | | | | |

2-parts composition 1:34 for 100 L acidic concentrate, about 23L(33.623 kg) SLURRY (solids+water+acid)

| | NaCl, | KCl, | CaCl2 × 2H2O, | MgCl2 × 6H20, | CH3COOH, | | Glucose × H2O, | Water, | KCl, | MgCl2 × 6H2O, | CH3COOH, |
|---|---|---|---|---|---|---|---|---|---|---|---|
| | kg | kg | kg | kg | kg | Water, L | kg | L | kg | kg | kg |
| Type | Acidic precursor | | | | | | Glucose precursor | | | | |
| Concen- | 0.722 | 0.002 | 0.026 | 0.011 | 0.021 | 0.155 | 0.132 | 0.055 | 0.016 | 0.0014 | 0.00017 |
| trate 1 | | | | | | | | | | | |
| Concen- | 0.722 | 0.002 | 0.022 | 0.011 | 0.021 | 0.156 | 0.132 | 0.055 | 0.016 | 0.0014 | 0.00017 |
| trate 2 | | | | | | | | | | | |
| Concen- | 0.722 | 0.011 | 0.026 | 0.011 | 0.021 | 0.150 | 0.132 | 0.055 | 0.016 | 0.0014 | 0.00017 |
| trate 3 | | | | | | | | | | | |
| Concen- | 0.722 | 0.011 | 0.022 | 0.011 | 0.021 | 0.152 | 0.132 | 0.055 | 0.016 | 0.0014 | 0.00017 |
| trate 4 | | | | | | | | | | | |
| Concen- | 0.722 | 0.020 | 0.026 | 0.011 | 0.021 | 0.148 | 0.132 | 0.055 | 0.016 | 0.0014 | 0.00017 |
| trate 5 | | | | | | | | | | | |
| Concen- | 0.722 | 0.020 | 0.022 | 0.011 | 0.021 | 0.148 | 0.132 | 0.055 | 0.016 | 0.0014 | 0.00017 |
| trate 6 | | | | | | | | | | | |

2-parts composition 1:34 for 3.429 L acidic concentrate, about 0.79 L(1.153 kg) SLURRY 1 patient needs about 1.153 kg slurry (1:34) during a 5-hour dialysis (dialysate flow about 0.4 L/min)

| | NaCl, | KCl, | CaCl2 × 2H2O, | MgCl2 ×6H20, | CH3COOH, | | Gluc. × H2O, | | KCl, | MgCl2 × 6H2O, | CH3COOH, |
|---|---|---|---|---|---|---|---|---|---|---|---|
| | kg | kg | kg | kg | kg | Water, L | kg | Water, L | kg | kg | kg |
| Type | Acidic precursor | | | | | | Glucose precursor | | | | |
| Concen- | 21.069 | 0.048 | 0.772 | 0.315 | 0.626 | 4.533 | 3.850 | 1.600 | 0.474 | 0.041 | 0.005 |
| trate 1 | | | | | | | | | | | |
| Concen- | 21.069 | 0.048 | 0.643 | 0.315 | 0.626 | 4.563 | 3.850 | 1.600 | 0.474 | 0.041 | 0.005 |
| trate 2 | | | | | | | | | | | |
| Concen- | 21.069 | 0.309 | 0.772 | 0.315 | 0.626 | 4.363 | 3.850 | 1.600 | 0.474 | 0.041 | 0.005 |
| trate 3 | | | | | | | | | | | |
| Concen- | 21.069 | 0.309 | 0.643 | 0.315 | 0.626 | 4.442 | 3.850 | 1.600 | 0.474 | 0.041 | 0.005 |
| trate 4 | | | | | | | | | | | |
| Concen- | 21.069 | 0.570 | 0.772 | 0.315 | 0.626 | 4.302 | 3.850 | 1.600 | 0.474 | 0.041 | 0.005 |
| trate 5 | | | | | | | | | | | |
| Concen- | 21.069 | 0.570 | 0.643 | 0.315 | 0.626 | 4.322 | 3.850 | 1.600 | 0.474 | 0.041 | 0.005 |
| trate 6 | | | | | | | | | | | |

1. A precursor of an acidic dialysis concentrate comprising two separately provided partial concentrates are formed from the constituents of the acidic dialysis concentrate, wherein only the first partial concentrate contains glucose, while the second partial concentrate comprises other constituents needed for the acidic dialysis concentrate.

2. The precursor of the acidic dialysis concentrate according to claim 1 wherein the first partial concentrate further comprises water, an acidic agent, and a salt selected from the group consisting of KCl, MgCl₂, CaCl₂, combinations thereof, in a slurry state.

3. The precursor of the acidic dialysis concentrate according to claim 2 wherein an amount of the salt is selected such that the salt is completely soluble in a saturated glucose solution at a temperature of about 5° C., wherein the glucose may remain in the undissolved state.

4. The precursor of the acidic dialysis concentrate according to claim 1 wherein the first partial concentrate is adjusted with acetic acid to a pH of 2 to 3.2.

5. The precursor of the acidic dialysis concentrate according to claim 1 wherein a ratio of the solids to water in the first partial concentrate is 60-64% by wt.

6. The precursor of the acidic dialysis concentrate according to claim 1 wherein the second partial concentrate contains sodium chloride and/or potassium chloride and/or calcium chloride and/or magnesium chloride or respective hydrates, water, and second acidic agent.

7. The precursor of the acidic dialysis concentrate according to claim 6 wherein the second partial concentrate is present in a slurry state in which constituents of the sodium chloride remain in the undissolved state.

8. The precursor of the acidic dialysis concentrate according to claim 6 wherein a ratio of solid to water in the second partial concentrate is about 71-82% by wt.

9. The precursor of the acidic dialysis concentrate according to claim 2 wherein an acidic agent concentration in liquid phase is below 10%.

10. The precursor of the acidic dialysis concentrate according to claim 1 wherein a weight ratio of the first to the second partial concentrate is 4.5 to 5.1:1 and a volume ratio is 3.2 to 3.6:1.

11. The precursor of the acidic dialysis concentrate according to claim 1 wherein the two partial concentrates are received in a container having two separated chambers.

12. The precursor of the acid dialysis concentrate according to claim 1 wherein the glucose being glucose monohydrate.

13. The precursor of the acid dialysis concentrate according to claim 2 wherein the acidic agent being acetic acid.

14. The precursor of the acid dialysis concentrate according to claim 6 wherein the second acidic agent being acetic acid.

15. The precursor of the acidic dialysis concentrate according to claim 4 wherein the pH is 3.0.

16. The precursor of the acidic dialysis concentrate according to 6 wherein a second acidic agent concentration in liquid phase is below 10%.
